(19) United States
(12) Patent Application Publication
(10) Pub. No.: US 2011/0306089 A1
(43) Pub. Date: Dec. 15, 2011

(54) THERMOPHILIC METHANOGENIC CONSORTIUM FOR CONVERSION OF CELLULOSIC BIOMASS TO BIOENERGY

(76) Inventors: Kevin R. Sowers, Baltimore, MD (US); Harold D. May, Charleston, SC (US); Chanlan Chun, Charleston, SC (US); Chrisopher William Marshall, Charleston, SC (US)

(21) Appl. No.: 13/027,105

(22) Filed: Feb. 14, 2011

Related U.S. Application Data
(60) Provisional application No. 61/304,140, filed on Feb. 12, 2010.

(51) Int. Cl. C12P 39/00; C12M 1/00; C12N 1/20

(52) U.S. Cl. 435/42; 435/252.1; 435/289.1

(57) ABSTRACT

A system for the efficient conversion of plant biomass to methane is provided, where the conversion includes use of a thermophilic methanogenic consortium containing a cellulolytic thermophile, an acetate-oxidizing thermophile and a thermophilic methanogen, the combination of which hydrolyzes hexoses and pentoses, oxidizes acetate and provides a hydrogen sink, to convert plant biomass to the theoretical limit of bioenergy.

### CROSS REFERENCE TO RELATED APPLICATION

This application claims the benefit of priority of U.S. Patent Application No. 61/304,140 filed Feb. 12, 2010. The disclosure of such foregoing application is hereby incorporated herein by reference in its respective entirety, for all purposes, and the priority of such application is hereby claimed under the provisions of 35 U.S.C. §120.

### FIELD OF THE INVENTION

The present invention relates to a thermophilic microbial consortium for conversion of biomass to methane with minimal waste and methods of using the same. Optionally, the consortium may be coupled to an electromethanogenic electrochemical cell to further maximize the efficiency of the conversion to methane.

### DESCRIPTION OF THE RELATED ART

Currently, the world depends heavily on a continuing supply of fossil fuels to meet energy demands. However, energy sources such as fossil fuels, coal, oil and natural gas, are non-renewable sources and their supply is diminishing.

Biofuels are a desirable alternative to fossil fuels and their use can help reduce the world's dependence on imported fossil fuels. Use of biofuels can provide relief with regard to the problems of limited fossil fuel resources and also detrimental effects on the environment resulting from use of fossil fuels.

Widespread recognition that natural gas reserves and infrastructure in the United States are extensive has led to the New Alternative Transportation to Give Americans Solutions Act of 2009, (HR1835 and S1408 of the 111^{th} Congress), to support a dramatic increase in the use of natural gas, particularly for transportation. In addition, the U.S. Department of Energy (DOE) just announced the award of $34 million to customers and government agency partners of Clean Energy Fuels Corp. to offset the cost of more than 800 new clean-burning natural gas-powered taxis, shuttle buses, refuse trucks and heavy-duty trucks. The energy density for liquefied natural gas (LNG) is 54 kJ/g versus 46, 44 and 30 kJ/g for diesel, gasoline and ethanol, respectively, so LNG would offset 44% more petroleum imports than an equivalent volume of ethanol (Drapcho, C. M., et al. Biofuels Engineering Process Technology. McGraw Hill (2008).). Although the increased use of U.S. reserves of natural gas can reduce the import of petroleum, this energy source is finite and its combustion product, carbon dioxide, contributes to the carbon footprint and global warming.

Ethanol has been proposed as one potential alternative fuel. In the United States, ethanol production has largely focused on corn as the source material. Ethanol derived from corn has been described as sustainable, green and environmentally friendlier than gasoline. However, in actual use, ethanol has drawbacks and potentially possesses a larger carbon footprint than originally thought, in full consideration of the use of fossil fuels in the production of ethanol. Even production of huge amounts of corn would be unlikely to meet the United States' fuel consumption needs. Additionally, growth of such large crops requires additional considerations of soil depletion, agricultural wastes and pollutants.

In production of ethanol, a large amount of water is required, and treatment costs of the water must also be factored in. Both production and use of ethanol can result in CO₂ release and can actually further contribute to the buildup of greenhouse gases. In addition to CO₂ release, ethanol combustion can result in byproducts such as aldehyde and can also contribute to the production of ground level ozone. Incomplete combustion of ethanol can result in carbon monoxide production.

Supply of ethanol to users must also consider the potential problems of transport and the potential for water contamination.

Biomethane has several advantages over many other biofuels currently under development. Biomethane is non-corrosive and can be transported in extant infrastructure; it has a high energy density and functions efficiently in existing vehicles. The U.S. Congress and industry are promoting the development of more methane powered vehicles to offset petroleum imports. Biomethane is self-distilling, renewable, sustainable, and carbon neutral, but it has not been developed for production from the next generation dedicated energy crops such as switchgrass.

Development of improved methods for increasing the production of methane would further support a shift of the world's transportation fleet to this fuel and it would have the added benefit of being sustainable, renewable and carbon neutral. Methane gas has several advantages: 1) it has a low solubility, which facilitates fuel collection without distillation; 2) unlike ethanol and hydrogen, which inhibit biomass conversion as they accumulate, methane does not inhibit the proposed process leading to more efficient biomass conversion to fuel (>86%); 3) it is readily distributed by an extant infrastructure used to transport natural gas without corrosion issues associated with ethanol and hydrogen; 4) methane generation could theoretically be maximized to generate no hazardous wastes and only minimal non-fermentable waste products; 5) lower-cost compression methods for liquefying methane are currently under development; 6) the gas can be used directly for transportation in internal combustion engines with minimum modification, and 7) energy can more readily be stored as methane than hydrogen and biomethane can be used as a source for hydrogen or methanol production (Ozkan, U., 2009, Design of Heterogeneous Catalysts: New Approaches based on Synthesis, Characterization and Modeling. Wiley; Rigpheil, M., et al., p. 223-233. In J. Wall (ed.), Bioenergy. ASM Press, Washington D.C., (2008); Sowers, K. R. Methanogenesis, p. 265-286. In L. Schaechter, et al.(ed.), Encylopedia of Microbiology, vol. 5. Elsevier, Oxford (2009) (http://www.inl.gov/lng/factsheets/liquefaction.pdf).

Gasoline spark ignition engines are the least efficient (16%) of current transportation technologies, whereas efficiency with methane in the same engine improves to 22% and hybrid hydrogen fuels cells are 50% efficient. The current limitation on promoting hydrogen for transportation is that most hydrogen comes from thermocatalytic reformation of natural gas. Like natural gas, biomethane offers the option of direct combustion in standard internal combustion engines for transportation or electrical generation, or thermocatalytic conversion to hydrogen for use in fuel cells. The process is slightly endothermic, so only a small fraction of the energy yield is lost from energy input (CH₄→C+2H₂ΔH^{o}=75.6 kJ/mol CH₄). However, unlike natural gas, combustion of biomethane does not result in a net release of carbon dioxide into the atmosphere.

Thermodynamic calculations show that 86% of the energy available in cellulose (glucose equivalents) is captured in methane versus only 33% as hydrogen (Schink, B. 2008. Energetic aspects of methanogenic feeding webs. In Bioenergy. J. Wall et al. (eds). 171-178). The latter case is due to the “Thauer” thermodynamic limit for hydrogen production of 4 mols per mol of glucose. The remainder of the energy is trapped in acetate, which cannot be used as a fuel. The yield for ethanol, 92%, is somewhat higher than that for methane, but ethanol dissolves in the aqueous medium and requires energy intensive separation procedures such as distillation. Furthermore, due to toxicity the highest ethanol concentrations observed in a fermentation broth is ˜12%, requiring even more energy to distill and concentrate the alcohol to a suitable level for use in an internal combustion engine. Butanol, another fermentation product being considered as a biofuel, reaches even lower concentrations in fermentation broth (˜5%). Estimates for producing methane from the next generation of dedicated energy crops such as switchgrass have not been published, but estimates for producing methane from whole sugar cane have been reported at 80% recovery of the energy content (Chynoweth, D. P., et al. Biomass Bioenergy (1993) 5:95-111; van Haandel, A. C. Water Sci. Technol. (2005) 52:49-57). Van Haandel (van Haandel, A. C. Water Sci. Technol. (2005) 52:49-57) has estimated that only 40% of the energy content of cane is recovered in ethanol.

There therefore remains a need in the art for more efficient sources and methods for producing biofuel, particularly biomethane.

Biomass for conversion into fuel is generally produced in two forms: carbohydrate materials containing starch and sugars, and lignocellulosics consisting of heterogeneous woody materials. The carbohydrate materials are more readily transformed biologically into biofuels. However, carbohydrates represent only a fraction of the total plant biomass and they are usually produced on valuable cropland that could be dedicated to food production. The carbohydrate biomass also comes from plants that require intense farming practices including the high use of fertilizer and water, which adds to their production costs and further increases their environmental impact. In contrast, lignocellulosics can be harvested from the waste plant material of food crops and forestry, or they can be grown as a dedicated energy crops on land ordinarily considered marginally beneficial. Some of the plants receiving the most consideration for development are switchgrass (Panicum virgatum), silver grass or miscanthus (Miscanthus giganteus), poplar (Populus spp.), and willow (Salix spp.). Among these switchgrass has the lowest production costs and great potential to be grown in most habitats in the United States. For example, in the southeast U.S., studies have shown that 5.4 to 7.2 dry MT of switchgrass can be grown per acre versus 2.3 MT of hay (Bransby, D. 2005. Switchgrass Profile. Available from hyper text transfer protocol bioenergy.ornl.gov/papers/misc/switchgrass-profile.html). Projections for near-term total production in the US have been estimated at 226 million dry MT per year, enough to theoretically produce 19.7 billion gallons of ethanol (Perlack, R. D., et al. (2005) Biomass Feedstock for a Bioenergy and Bioproducts Industry: The Technical Feasibility of a Billion-ton Annual Supply. U.S. Department of Energy and U.S. Department of Agriculture.). Since dedicated energy crops have not been produced yet on a large scale, future projections will have to be updated, but the U.S. DOE and USDA have estimated that by the mid-21st century 342 million dry MT of energy crops could be produced potentially in the country (Perlack, R. D., et al.)).

To date, pretreatment of lignocellulosics is necessary to maximize biofuel yield. Lignocellulose consists of cellulose, hemicellulose and lignin. Lignin is recalcitrant to most biological degradation and an effective process has not yet been developed for complete bioconversion of this feedstock to biofuel. Furthermore, the lignin component and the overall integration of the three components of lignocellulose ordinarily render most of the cellulose and hemicellulose inaccessible to hydrolytic microorganisms or their enzymes. For this reason, pretreatment of the biomass prior to hydrolysis is deemed necessary and several processes are under development (Drapcho, C. M., et al. Biofuels Engineering Process Technology. McGraw Hill (2008).). Hydrolysis with concentrated or dilute sulfuric acid hydrolysis are amongst the most commonly considered processes and are perhaps closest to commercialization, but they require neutralization and high energy input to achieve the requisite temperatures (120° C. to >200° C. for the dilute acid process).

One approach for biofuel production is microbe engineering. Mesophilic (38° C.) tricultures of a cellulolytic fungus, a hydrogenotrophic methanogen and an aceticlastic methanogen (Mountfort, D. O., et al. (1982) Appl. Environ. Microbiol. 44:128-134; Nakashimada, Y., K. et al. Biotechnol. Lett. (2000) 22:223-227) were examined in the past. These cultures produced methane and improved the rates of cellulose hydrolysis, but the rates of metabolism were slower due to mesophilic temperatures and production of inhibitory byproducts such as ethanol and lactate. In addition, these cultures did not simultaneously consume pentoses.

Thermophilic Clostridium thermocellum and Methanothermobacter thermoautotrophicus were tested in co-culture with cellulose (Weimer, P. J., et al. Appl. Env. Microbiol. (1977) 33:289-297). The latter co-culture produced methane at 60° C. but did not consume pentoses, inhibitory byproducts such as ethanol and lactate formed, and the methanogen's rate of metabolism was slowed significantly at this temperature 60° C.

Thermophilic C. thermocellum and M. thermoautotrophicus were evaluated in a tri-culture with Methanosarcina MP (Smiti, N. et al., FEMS Microbiology Lett. 35 (1986) 93-97.), where the aceticlastic activity of Methanosarcina MP degraded acetate, and acted to produce methane at 60° C. However, the activity of this tri-culture was only demonstrated with cellulose, it was not demonstrated at temperatures greater than 60° C. and the triculture required addition of methanol to achieve complete acetate degradation.

There therefore remains a need in the art for more efficient methods of production of biofuels, particularly biomethane, using lignocellulosics as a sustainable feedstock. Such production would result in a sustainable, green method for obtaining alternatives to traditional fossil fuels.

### SUMMARY OF THE INVENTION

The present invention relates to a system and methods using a thermophilic microbial consortium for conversion of biomass to methane with minimal waste.

In one aspect, the invention relates to a thermophilic microbial consortium for conversion of cellulosic or lignocellulosic biomass to methane, the consortium including a cellulolytic thermophile, an acetate-oxidizing thermophile effective to oxidize acetate to carbon dioxide and hydrogen, and a hydrogen-utilizing thermophilic methanogen.

In another aspect, the invention relates to a system for the conversion of cellulosic or lignocellulosic biomass to methane, the system including a thermophilic microbial consortium comprising a cellulolytic thermophile, an acetate-oxidizing thermophile effective to oxidize acetate to carbon dioxide and hydrogen, and a hydrogen-utilizing thermophilic methanogen, and an electromethanogenic electrochemical cell.

In a further aspect, the invention relates to a method of converting lignocellulosic biomass to methane, comprising exposing the biomass to a thermophilic microbial consortium comprising a cellulolytic thermophile, an acetate-oxidizing thermophile effective to oxidize acetate to carbon dioxide and hydrogen, and a hydrogen-utilizing thermophilic methanogen, under conditions effective for microbial action on the biomass to produce lignin, CO₂ and CH₄.

Still another aspect of the invention relates to a method comprising microbial conversion of cellulosic or lignocellulosic biomass to methane, wherein the microbial conversion comprises microbial action by a cellulolytic thermophile, an acetate-oxidizing thermophile and a hydrogen-utilizing methanogenic thermophile.

Other aspects, features and embodiments of the invention will be more fully apparent from the ensuing disclosure and appended claims.

### BRIEF DESCRIPTION OF THE DRAWINGS

FIG. 1 is a diagram of an upflow anaerobic digestor with C. saccharolyticus, Thermincola ferriacetica and M. thermoautotrophicus.

FIG. 2 is a diagram of an upflow anaerobic digestor with C. saccharolyticus and M. thermoautotrophicus and an electromethanogenic cell attached to further convert CO₂ to methane and purify the fuel stream.

FIG. 3 is a flowchart of a methanogenic tri-culture of the invention and its cellulolytic/methanogenic pathway.

FIG. 4 is a graph taken from prior art reference Mountfort et al. (Mountfort, D. O., et al., Appl. Environ. Microbiol. (1982) 44:128-134) demonstrating increased cellulose degradation by mesophilic methanogenic tri-culture.

FIG. 5 is a graph showing the methane production by M. thermoautotrophicus incubated with C. saccharolyticus with cellulose, as described in Example 1. Estimation of expected H₂ was based on the measured CH₄.

FIG. 6 is a graph showing the hydrogen and methane profile in the coculture of C. saccharolyticus and M. thermoautotrophicus grown with 2.5 g/L cellulose, as described in Example 1.

FIG. 7 is a graph showing the production of hydrogen, or hydrogen equivalents produced as methane, from C. saccharolyticus alone, the co-culture of M. thermoautotrophicus incubated with C. saccharolyticus (Example 1), and the tri-culture of M. thermoautotrophicus, C. saccharolyticus and T. ferriacetica (Example 3) when incubated with 2.5 g of cellulose powder.

FIG. 8 is a graph showing the methane production by M. thermoautotrophicus incubated with T. ferriacetica and acetate, as described in Example 1.

FIG. 9 is a graph comparing hydrogen production in the coculture of C. saccharolyticus and M. thermoautotrophicus with that in the monoculture of C. saccharolyticus grown with cellulose, as described in Example 2. Arrows presents when 0.1 g/L of cellulose was added to both cultures in a semi-batch mode.

FIG. 10 is two graphs showing distribution of fermentation products in the monoculture (FIG. 10A) and the coculture (FIG. 10B). Data are shown from culture experiments of FIG. 9.

FIG. 11 is a graph comparing hydrogen production in the coculture of C. saccharolyticus and M. thermoautotrophicus with that in the monoculture of C. saccharolyticus when grown with 5 g/L untreated switch grass in batch mode, as described in Example 5.

FIG. 12 provides optical micrographs of the monoculture of C. saccharolyticus (12A and 12B) and the coculture of C. saccharolyticus and M. thermoautotrophicus (12C and 12D) after Gram staining, as described in Example 6. Cultures were grown with 2.5 g/L cellulose for 3 days.

### DETAILED DESCRIPTION OF THE INVENTION, AND PREFERRED EMBODIMENTS THEREOF

In response to the national mandate to discover new domestic sources of energy, reduce the national dependence on imported fossil fuels, and mitigate the threat of global warming, the present invention provides a new bioenergy technology that enhances the conversion of cellulosic or lignocellulosic biomass to biofuel. The invention provides a new, more productive process whereby methanogenesis is used to more effectively hydrolyze cellulosic or lignocellulosic biomass from renewable, non-food energy crops. The conversion of cellulosic or lignocellulosic biomass to biomethane is achieved by use of an optimized thermophilic microbial consortium. The consortium provides conversion of cellulosic or lignocellulosic biomass to biofuel through maximization of interspecies hydrogen and acetate transfer between thermophilic species of cellulolytic/hemicellulolytic bacteria and methanogenic bacteria.

“Biomass” as used herein refers to a renewable energy source which is biological material from living or recently living organisms. As used herein, the term “biomass” may include lignocellulosic biomass and/or cellulosic biomass.

The invention provides an optimized thermophilic microbial consortium comprising: 1) a cellulose and hemicellulose hydrolyzing cellulolytic thermophile, 2) an acetate-oxidizing thermophile, and 3) a hydrogen-utilizing thermophilic methanogen. The optimized thermophilic microbial consortium achieves conversion of cellulosic biomass to bioenergy with a yield that is nearly 86% of theoretical. The proposed system leverages the fast metabolism of thermophilic bacteria, resists fouling or contamination that reduces efficient conversion of biomass to energy, and produces a self-distilled product that does not inhibit cellulosic fermentation the way that ethanol or H₂ does.

Overall, the addition of the acetate-oxidizing thermophile and thermophilic methanogen will result in increased rates of cellulose/hemicellulose hydrolysis and greater yields of a biofuel, methane, which has an extant infrastructure and is being considered by the U.S. Congress and industry for increased use as a transportation fuel.

The optimized thermophilic microbial consortia and methods of using the consortia presented herein improve the energy yield from cellulosic biomass, identify the effect of the consortium on hydrolytic and fermentative gene expression, and maximize cellulose hydrolysis and biomethane production in a continuous flow bioreactor, as compared to previously known methods. The invention provides a highly efficient, large-scale conversion of cellulosic biomass to biomethane fuel with optimized carbon and electron flow for such conversion.

Use of microbial consortia in generation of biofuels is an evolving technology. Currently known microbial consortia suffer from limitations such as excessive CO₂ production and/or generation of other byproducts which may be toxic. Furthermore the byproducts of known microbial consortia may be limited in their use, e.g. only being useful in situ.

In one embodiment the present invention provides an optimized thermophilic microbial consortium containing a cellulose/hemicellulose hydrolyzing fermentor that produces acetate, CO₂ and H₂, an acetate-oxidizing hydrogen producing syntrophic bacterium and a hydrogen utilizing methanogen is used to achieve conversion of cellulosic plant material to the maximum theoretical yield of methane (CH₄) and carbon dioxide (CO₂).

As utilized herein, the terms “consortium,” “consortia,” “microbial culture” and tri-culture” all refer to a group of microorganisms combined so that the microorganisms work in a collaborative manner in order to obtain maximum biomethane from lignocellulosics as a feedstock. In one embodiment of the present invention the consortium includes all thermophilic microorganisms. In another embodiment of the invention, the consortium includes a cellulose and hemicellulose hydrolyzing cellulolytic thermophile, an acetate-oxidizing thermophile, and a hydrogen-utilizing thermophilic methanogen.

In order to maximize the methane production, the biogases resulting from action of the thermophilic microbial consortium are passed through a secondary reactor containing an electromethanogenic electrochemical cell, which is effective to convert carbon dioxide to methane. By using thermophilic microorganisms for the entire process the conversion rate is accelerated several-fold compared with mesophilic processes, energy diverting fouling reactions due to contamination are avoided, and the overall fermentation yields only one product: biomethane. The net yield of the process is the maximum theoretical yield of cellulosic plant material to biomethane with no toxic bioproducts and no release of CO₂. The release of carbon is a net zero with the fermentation process described and is negative when combined with electromethanogenesis. The only waste product is lignin, which can be harvested for additional energy by combustion in a boiler to heat the system or generate electricity.

The invention therefore provides a thermophilic microbial consortium in which, unlike fermentative monocultures commonly used for ethanol or hydrogen production, nearly all usable energy is harvested from the organic biomass and converted to the maximum theoretical yield of biofuel in a single process. This technology can be scaled up and transferred to the marketplace quickly due to the infrastructure already in place for biomethane and natural gas.

In one embodiment of the invention, all microorganisms used in the consortia are thermophilic microorganisms. “Thermophilic,” as used herein means that each microorganism is active in the production of methane by the consortium at temperatures of about 60° F. or above. In another embodiment each microorganism is thermophilically active at a temperature of at least about 65° C. In a preferred embodiment each microorganism is thermophilically active at a temperature of at least about 70° C. In a particularly preferred embodiment each microorganism are thermophilically active at a temperature of at least about 75° C. In another embodiment each microorganism is thermophilically active at a temperature of at least about 50° C. or at least about at least about 55° C. A thermophilic consortium will maximize hydrolysis of cellulosics and the production rate of methane.

Thermophilic bioprocessing at temperatures above 60° C. will have several benefits. In general, process kinetics double for every 10-degree increase in temperature and cellulose degradation increases dramatically with temperature (Lynd, L. R., Microbiol. Mol. Biol. Rev. (2002) 66:506-577.). Cellulose hydrolysis is generally a limiting factor for production of cellulosic biofuels at mesophilic temperatures. Accordingly, the overall footprint of a bioreactor can be reduced because cellulolytic metabolism proceeds at a faster pace.

Furthermore, many industrial processes are operated at elevated temperatures and waste heat from the plant is often available for use. Excess heat generated during the 60° C. SAA pretreatment of cellulosic biomass would be adequate to heat most of the process and the fermentation process itself will also generate excess heat.

Another significant advantage of using high temperature is the resistance to fouling due to bacterial contamination that would divert energy into non-useful products (Skinner, K. A., et al. J. Ind. Microbiol. Biotechnol. 31:401-408 (2004); van Groenestijn, J. W., et al. Inter. J. Hyd. Energy (2002) 27:1141-1147.). This also minimizes or negates the need for sterilization to maintain a defined inoculum, which would be cost prohibitive.

And finally, thermophilic metabolic pathways tend to produce a limited range of products; therefore the metabolism can be more readily directed towards the desired product, e.g. hydrogen or methane.

Cellulosic biomass is widely considered to be a viable source of sustainable, carbon neutral feedstock for the production of biofuels that will ultimately reduce the national reliance on fossil fuels (Lynd, L., et al., Nat. Biotechnol. (2008) 26:169-172). Anaerobic fermentation is the most common method of converting biomass to biofuels and among the approaches currently under investigation to increase the rate of cellulose degradation is bioconversion at higher temperature (Lynd et al., Microbiol. Mol. Biol. Rev. (2002) 66:506-577).

Therefore, in one embodiment the thermophilic microbial consortium contains a cellulolytic thermophile which is effective to hydrolyze both cellulose and hemicellulose at high temperatures.

Clostridium thermocellum is well known for its high rates of cellulose hydrolysis (Lynd, L. R., Microbiol. Mol. Biol. Rev. (2002) 66:506-577.). However, its optimal growth temperature is only 60° C., it is unable to consume pentoses generated from hemicellulose, and it produces ethanol, acetate, lactate and sometimes butyrate in addition to H₂ and CO₂. This inefficient conversion of cellulosic biomass has led to the search for a thermophilic bacterium with a higher temperature optimum, broader substrate range, and a fermentation pathway with fewer products.

The most thermophilic of the cellulolytic anaerobes have a Tₒₚₜ of 70-75° C. and are capable of hydrolyzing a broad range of carbohydrates, particularly the two main components of plant biomass: cellulose and hemicellulose (Reynolds, P. H. S., et al., (1986) Appl. Environ. Microbiol. 51:12-17; Svetlichnyi, V. A, (1990) Microbiol. 59:598-604; van de Werken, H. J. G. (2008) Appl. Environ. Microbiol. 74:6720-6729; Yang, S.-J., et al., (2009) Appl. Environ. Microbiol. 75:4762-4769).

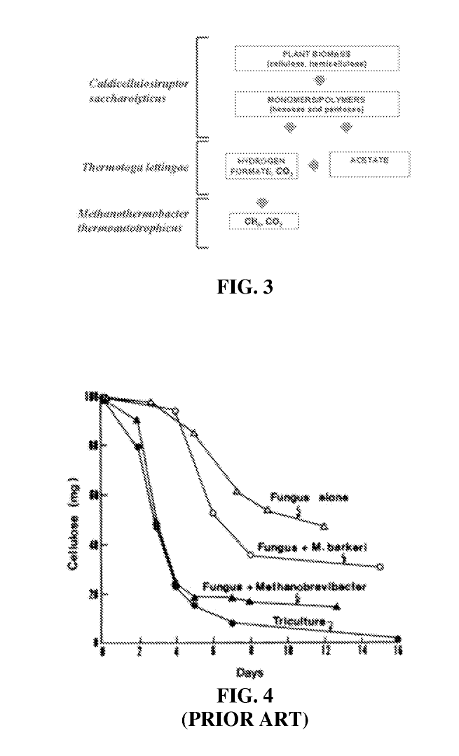

This group of microorganisms includes Caldicellulosiruptor saccharolyticus (C. saccharolyticus; DSM 8903, ATCC 43494) and Caldicellulosiruptor bescii (C. bescii, strain 6725; formerly identified as Anaerocellum thermophilum DSM 6725), closely related bacterial species that are unique in their ability to concurrently hydrolyze cellulose/hemicellulose and convert the polymers into only acetate, CO₂ and H₂ (FIG. 3). Both have the ability to hydrolyze crystalline forms of lignocellulose.

The cellulases of these organisms are of interest to both the bioethanol and biohydrogen industries. However, the hydrogen and fatty acids produced by these organisms inhibit complete hydrolysis and fermentation of cellulose.

C. saccharolyticus is an extreme thermophile with a Tₒₚₜ of 70° C., and which is capable of growing on a wide variety of plant polymers including cellulose, hemicellulose, starch and pectin (Ivanova, G., et al., Inter. J. Hydrogen Energy (2008) 33:6953-6961; Ivanova, G., et al., Inter. J. Hydrogen Energy (2009) 34:3659-3670; van de Werken, H. J. G., et al., Appl. Environ. Microbiol. (2008) 74:6720-6729; Yang, S.-J., et al., Appl. Environ. Microbiol. (2009) 75:4762-4769). Furthermore, it simultaneously uses the hexoses and pentoses that are generated from hydrolysis of cellulosic biomass. It is particularly attractive as a catalyst for producing hydrogen from cellulose since it yields nearly 4 mol of H₂ per mol of glucose, thereby reaching the “Thauer” or thermodynamic limit (C₆H₁₂O₆→2CH₃COO⁻+2HCO₃⁻+4H₂+4H⁺). While generating H₂ it also produces acetate, but it does not produce alcohols or other fatty acids unless the hydrogen partial pressure becomes too high, in which case lactate is formed and the cellulosic fermentation is eventually inhibited. The acetate produced by the organism also eventually inhibits fermentation and growth.

C. saccharolyticus is very closely related to C. bescii, another extreme thermophile with a Tₒₚₜ of 75° C., and which is capable of hydrolyzing cellulose and hemicellulose and simultaneously consuming the hexoses and pentoses generated from these polymers. C. bescii is also a good producer of hydrogen and it does not produce alcohols to a significant level. It also produces acetate and lactate, which accumulate and eventually inhibit its growth and ability to consume cellulose.

C. saccharolyticus and C. bescii both can degrade switchgrass, a relatively high lignin grass, or other low lignin grasses such as Bermuda, without pretreatment, but only C. bescii will grow on higher lignin plants such as poplar. The ability to hydrolyze cellulosic biomass presents a significant advantage in the production of bioenergy from cellulose since the pretreatment costs are avoided or minimized and other inhibitory compounds are often produced during harsh pretreatments.

While pretreatment of biomass prior to hydrolysis by a cellulolytic thermophile is not necessary, in a further embodiment of the invention such pretreatment is performed. In one embodiment the pretreatment is soaking the biomass in aqueous ammonia (SAA) (Isci, A., et al., Appl. Biochem. Biotechnol. (2008) 144:69-77; Kim, T. H., and Y. Y. Lee, Appl. Biochem. Biotechnol. (2005) 121-124:1119-1132.). This process may be performed at room or moderate temperature (60° C.) and the ammonia may be volatilized and recycled, which eliminates the cost of chemicals. Up to 74% of the lignin is removed and can be burned to heat a boiler, and ˜90% of the cellulose and 50% of the hemicellulose are recovered. Research continues to achieve greater recovery of the hemicellulose from different feedstocks, but SAA is a suitable pretreament for switchgrass since the feedstock is composed primarily of cellulose (39.8% versus 18.4% hemicellulose).

In another embodiment the pretreatment is selected from concentrated sulfuric acid hydrolysis, lime treatment and steam explosion or any other pretreatment known to those skilled in the art, such as those methods described in Drapcho, et al., (2008) Biofuels Engineering Process Technology. McGraw Hill.

In monoculture C. saccharolyticus and C. bescii produce H₂ from carbohydrates at nearly the thermodynamic limit (4 mols H₂ per mol glucose), will hydrolyze cellulosic without pretreatment, and can simultaneously metabolize hexose and pentose sugars. However, in the monoculture the production and accumulation of acetate reduces the total yield of biofuel, and the production and accumulation of hydrogen, which is not energetically favorable, inhibits the overall rate of cellulose hydrolysis.

In order to overcome the limitations commonly associated with monoculture bioconversion of plant biomass, a consortium of the present invention was developed, wherein the cellulolytic thermophile is combined with additional microorganisms to address the accumulation of acetate and hydrogen. In one embodiment the invention provides a thermophilic tri-culture in which the cellulolytic thermophile is grown in thermophilic tri-culture with an acetate-oxidizing syntroph and a hydrogen-consuming methanogen. Such a thermophilic system maintains intermediates below inhibitory concentrations, which will dramatically improve the rate of cellulose hydrolysis and increase total energy yield due to the efficient conversion of all inhibitory fermentation intermediates into biomethane.

In one embodiment the cellulolytic thermophile is selected from Caldicelluosiruptor saccharolyticus and Caldicellulosiruptor bescii, as described in Table 1 below.

TABLE 1

| Cellulolytic | | | | |
|---|---|---|---|---|
| Thermophile | Substrate(s) | Product(s) | pH | Tₒₚₜ (° C.) |
| Caldicellulosiruptor | Cellulose, | Hydrogen, acetate | 7.2 | 70 |
| saccharolyticus | hemicellulose, | (sometimes | | |
| | xylose | lactate), CO₂ | | |
| Caldicellulosiruptor | Cellulose, | Hydrogen, acetate | 7.2 | 75 |
| bescii strain DSM | hemicellulose, | (sometimes | | |
| 6725 [formerly | xylose | lactate), CO₂ | | |
| identified as | | | | |
| Anaerocellum | | | | |
| thermophilum] | | | | |

Since the cellulolytic thermophiles of the invention are acetogenic, the accumulation of acetate will eventually inhibit the process before it achieves complete hydrolysis and fermentation. Accordingly, in one embodiment the invention provides a thermophilic microbial consortium in which the acetate is further metabolized.

There are two potential microbial processes for maintaining low steady state acetate levels. In one, aceticlastic methanogensis, acetate is cleaved to methyl and carboxyl groups. The methyl group is reduced to generate methane and the carboxyl group is oxidized to CO₂ via an exergonic reaction (ΔG^{o′}=−31.0 kJ/mol). There are two known genera of aceticlastic methanogens, Methanosarcina and Methanosaeta. Although thermophilic species of each genus have been described, they cannot grow at the >60° C. required by the cellulolytic thermophiles of the thermophilic microbial consortium of the invention.

The second microbial processes for maintaining low steady state acetate levels consists of two reactions, syntrophic acetate oxidation coupled to hydrogenotrophic methanogenesis. In syntrophic acetate oxidation, both methyl and carboxyl groups of acetate are oxidized to CO₂ coupled with proton reduction to generate H₂. This reaction is thermodynamically unfavorable (ΔG^{o′}=+104.6 kJ/mol) but can proceed if H₂-consuming methanogenesis (ΔG^{o′}=−135.6 kJ/mol) maintains a low dissolved hydrogen partial pressure. The overall reaction becomes exergonic with the same free energy (ΔG^{o}′=−31.0 kJ/mol) and net stoichiometry (CH₃COO⁻+H₂O→HCO₃⁻+CH₄) as aceticlastic methanogenesis.

Four acetate-oxidizing syntrophs have been described: Thermoacetogenium phaeum, Clostridium ultunense, strain AOR and Thermotoga lettingae. Only one of these, Thermotoga lettingae, has a growth temperature in a range of 50-75° and a Tₒₚₜ of about 65° C. (Balk, M., et al., Int. J. Syst. Evol. Microbiol. (2002) 52:1361-1368; Hattori, S., et al., J Biosci Bioeng (2001) 91:264-298), which is compatible with the Tₒₚₜ of C. saccharolyticus and C. bescii.

By creating a thermophilic microbial consortium of a cellulose fermenting, acetate producing cellulolytic thermophile with an acetate-oxidizing syntroph and a hydrogenotrophic methanogen, all of the biomass is efficiently converted first to hydrogen and carbon dioxide and then to methane (FIG. 3).

However, T. lettingae also consumes hexose and pentose sugars, which might inhibit acetate utilization due to catabolite repression. Catabolite repression has not been examined in T. lettingae, but it is likely that the organism will utilize sugars before acetate and that acetate consumption will not be constitutively expressed. In batch cultures complete conversion to methane occurs as long as the cellulose concentration is not too high. However, a two-stage fermentation may be required to avoid the accumulation of acetate and maximize the productivity of the system (see Examples 9 and 10).

The inventors recently discovered that another acetate-oxidizing thermophile, Thermincola ferriacetica, will oxidize acetate to hydrogen and carbon dioxide when grown syntrophically with a methanogen. Previously this microorganism was known only to link oxidation of acetate with reduction of iron (Zavarzina, D. G., et al. Extremophiles (2007) 11:1-7) on the anode of a bioelectrochemical cell (Marshall, C. W., et al. Energy Environ. Sci. (2008) 2:699-705). This microorganism will not grow with any hexose or pentose sugars, thus using T. ferriacetica eliminates the potential for catabolite repression due to the presence of sugars blocking the oxidation of acetate. It also grows at 65-70° C. and is therefore compatible with the cellulolytic thermophile and a thermophilic methanogen.

As described herein, T. ferriacetica has been shown to grow syntrophically with M. thermoautotrophicus with acetate as the sole electron donor and generate methane. Each microorganism has been shown to grow in the others’ medium. If acetate-oxidation by T. lettingae is not inhibited by the presence of carbohydrates or if T. ferriacetica will generate hydrogen syntrophically then it is likely that only a single stage bioreactor will be needed for the proposed system. T. ferriacetica offers one other experimental alternative; grow C. saccharolyticus with an acetate-oxidizing bacterium sans the methanogen in order to explicitly examine the affect of interspecies acetate transfer on C. saccharolyticus while it consumes cellulose.

Therefore, in one embodiment the thermophilic microbial consortium contains an acetate-oxidizing thermophile which is effective to oxidize acetogen at high temperatures. By such reverse acetogenesis, the acetate-oxidizing thermophile utilizes acetate as a starting product, producing CO₂ and H₂.

In one embodiment the acetate-oxidizing thermophile is selected from Thermatoga lettingae and Thermincola ferriacetica, as described in Table 2 below.

TABLE 2

| Acetate-oxidizing | | | | |
|---|---|---|---|---|
| Thermophile | Substrate(s) | Product(s) | pH | Tₒₚₜ (° C.) |
| Thermatoga | acetate | Hydrogen, CO₂ | 7.0 | 50-75 |
| lettingae | | | | |
| Thermincola | acetate | Hydrogen, CO₂ | 7.0-7.2 | 60-70 |
| ferriacetica | | | | |

As described in detail herein, both the action of the cellulolytic thermophile and the acetate-oxidizing thermophile result in H₂ accumulation and benefit from the presence of an H₂-consuming methanogen in order to maintain low dissolved hydrogen partial pressure within the reactor containing the consortium and in order to keep the reaction proceeding to completion. “Completion,” as described herein with regard to the reactor containing the tri-microbial thermophilic consortium is achieving maximum theoretical yield of methane from the cellulosic biomass starting product, without toxic byproducts and with only CO₂ produced.

Therefore, in one embodiment the thermophilic microbial consortium contains a thermophilic methanogen which is effective to act as a thermophilic hydrogen sink in the conversion of H₂ to methane at high temperatures.

In one embodiment the thermophilic methanogen is selected from Table 3 below:

TABLE 3

| Thermophilic Methanogen | Substrate(s) | Product(s) | pH | Tₒₚₜ (° C.) |
|---|---|---|---|---|
| Methanothermobacter | Hydrogen | methane, CO₂ | 7.2-7.6 | 65-70 |
| thermoautotrophicus | | | | |
| Methanobacterium thermoaggregans | Hydrogen | methane, CO₂ | 7.0-7.5 | 65 |
| Methanothermobacter defluvii | Hydrogen, | methane, CO₂ | 6.5-7.0 | 60-65 |
| | formate | | | |
| Methanothermobacter marburgensis | Hydrogen | methane, CO₂ | 6.8-7.4 | 65 |
| Methanothermobacter wolfei | Hydrogen, | methane, CO₂ | 7.0-7.7 | 55-65 |
| | formate | | | |

In another embodiment the thermophilic consortium is a high salt environment and the thermophilic methanogen is derived from a marine origin. In such embodiment the thermophilic methanogen is selected from Table 4 below:

TABLE 4

| Thermophilic | | | | |
|---|---|---|---|---|
| Methanogen | Substrate(s) | Product(s) | pH | Tₒₚₜ (° C.) |
| Methanotorris | Hydrogen, | methane, | 6.7 | 75 |
| formicicus | formate | CO₂ | | |
| Methanothermococcus | Hydrogen, | methane, | 6.0-7.0 | 60-65 |
| okinawensis | formate | CO₂ | | |
| Methanothermococcus | Hydrogen, | methane, | 6.5-7.5 | 65 |
| thermolithotrophicus | formate | CO₂ | | |

C. saccharolyticus, as an exemplary cellulolytic thermophile described herein, is capable of converting biomass to the hypothetical “Thauer” limit of 4 mols H₂ per mol glucose (Thauer, R., K. et al., Bacteriol. Rev. (1977) 41:100-180). However, in the absence of a hydrogen-sink the process will not achieve the hypothetical maximum because proton reduction is thermodynamically unfavorable under standard conditions. Growing a fermentative microorganism such as C. saccharolyticus with a hydrogen-utilizing methanogen, such as M. thermoautotrophicus, enables the cellulose fermentor to hydrolyze and ferment biomass to the theoretical limit by maintaining a dissolved hydrogen concentration of ca. 10-12 nM (Zinder, S., p. 128-206. In J. G. Ferry (ed.), Methanogenesis. Chapman and Hall, New York (1993)). This alone will result in a significant improvement in the conversion of cellulosic biomass to biofuel.

M. thermoautotrophicus ΔH is an extreme thermophile (Tₒₚₜ of 70° C.) that is very good at scavenging H₂ and using it to convert CO₂ to CH₄ (Smith et al., (1997) J. Bacteriol. 179:7135-7155.) By growing M. thermoautotrophicus or other thermophilic methanogens (Table 4) with C. saccharolyticus or C. bescii in co-culture it is possible to maintain a low partial pressure of hydrogen and avoid inhibition of fermentation due to the accumulation of hydrogen and subsequent production of lactate instead of acetate.

The accumulated acetate would be oxidized to hydrogen and carbon dioxide by action of the acetate-oxidizing thermophile in the presence of the methanogen, as described in detail above. The net products of the combined tri-culture are methane and carbon dioxide.

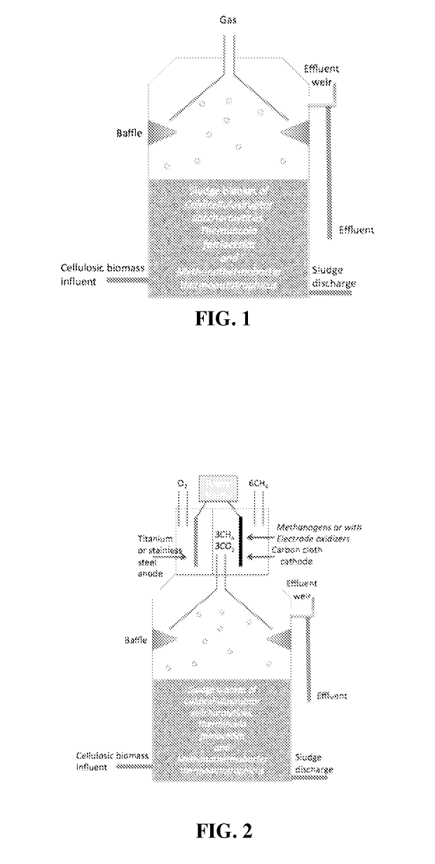

FIG. 1 illustrates the combination of an upflow anaerobic digestor inoculated with C. saccharolyticus, T. ferriacetica and M. thermoautotrophicus supplied with untreated or pretreated cellulosic biomass (e.g. ammonia-treated switchgrass). The tri-culture of microorganisms will produce granules of microbial biomass or grow as a biofilm on the surface of the cellulose particles, similar to what is formed in anaerobic treatment systems that process organic waste. The hydrogen consumption by the methanogen directs the fermentation pathway toward hydrogen production by C. saccharolyticus, and residual acetate that forms will be converted to methane and CO₂ by the syntroph functioning as an acetate-oxidizing hydrogen producer in the presence of the methanogen.

All of the microorganisms selected for the thermophilic microbial consortium of the invention can be grown in the same medium at neutral pH. A source of nitrogen could be a problem with some feedstocks such as nitrogen poor switchgrass. However, M. thermoautotrophicus can fix nitrogen and if an ammonia pretreatment of the lignocellulose is used, a common method (Isci, A., et al. Appl. Biochem. Biotechnol. (2008) 144:69-77), a small amount of the ammonia could be transferred to the fermentation vessel while the remainder is recycled for further biomass pretreatment. The thermophilic microbial consortium will function without treatment of the cellulosic biomass, but the ammonia pretreatment will be compatible with the triculture and may enhance conversion rates while avoiding the production of inhibitory compounds, the use of acids, high temperature, or neutralizing chemicals.

Because all of the chemical energy from the cellulosic biomass will be converted into substrates for methanogenesis, and there is no accumulation of potentially inhibitory intermediates, the thermophilic microbial consortium of the invention dramatically improves the process of cellulose conversion into biofuel. Mountfort et al. (Mountfort, D. O., et al., Appl. Environ. Microbiol. (1982) 44:128-134.) demonstrated that a mesophilic tri-culture improved cellulose hydrolysis (FIG. 4). The anaerobic tri-culture consisted of a cellulolytic fungus plus an aceticlastic methanogen and a hydrogenotrophic methanogen. However, that community lacked the advantages of thermophiles and C. saccharolyticus, by which the thermophilic microbial consortium of the invention provides a more effective method for hydrolyzing cellulose/hemicellulose and producing bioenergy in the form of methane.

Each of the cellulolytic thermophile, acetate-oxidizing thermophile, and thermophilic methanogen were selected, as described in detail herein, based on examination in pure culture and in co-culture to determine the role of each on the ability of the tri-culture to convert cellulose/hemicellulose to hydrogen and then methane. Example 1, set forth below, provides a co-culture of C. saccharolyticus and M. thermoautotrophicus that demonstrates methanogenesis through interspecies H₂ transfer between the C. saccharolyticus and the hydrogenotrophic methanogen M. thermoautotrophicus. Example 1 also reports a co-culture of M. thermoautotrophicus with T. ferriacetica that demonstrates that T. ferriacetica grows as an acetate-oxidizer in the presence of a hydrogen utilizing methanogen. Taken together, the results of these co-cultures provide support for using a tri-culture of C. saccharolyticus, T. ferriacetica and M. thermoautotrophicus at a thermophilic temperature of 70° C. to efficiently convert cellulosic biomass to biomethane and carbon dioxide, then convert the carbon dioxide to methane by electromethanogenesis.

Further, co-culturing C. saccharolyticus with M. thermoautotrophicus significantly improved the rate and yield of cellulose hydrolysis in comparison with a monoculture of C. saccharolyticus. Example 2 demonstrates that interspecies H₂ transfer has beneficial effects on cellulose hydrolysis by C. saccharolyticus.

Following tests of co-cultures, a tri-culture of C. saccharolyticus, M. thermoautotrophicus and T. lettingae/T. ferriacetica was performed as described in Example 3, which demonstrated the ability of the tri-culture to hydrolyze cellulose and produce methane. M. thermoautotrophicus drives the fermentation from cellulose→H₂/CO₂→CH₄ and perhaps cause a reduction in the production of acetate, but it is still likely that acetate will accumulate. T. lettingae converts the residual acetate to H₂ and CO₂, thus enabling the tri-culture to complete the fermentation by diverting most reducing equivalents to methane.

The net yield of this process is complete conversion of total COD from the cellulosic and hemicellulosic components of the plant biomass to biomethane with net zero release of carbon, negative release with electromethanogenesis, and lignin as the only waste product. This ability was reviewed with respect to crystalline cellulose and both untreated and pretreated cellulosic biomass from non-food crops such as switchgrass.

The co-culturing of C. saccharolyticus with M. thermoautotrophicus with untreated switchgrass as a substrate significantly improved the yield of hydrogen in comparison with a monoculture of C. saccharolyticus. Example 5 demonstrates that interspecies hydrogen transfer promotes greater hydrolysis of lignocellulosics.

Example 6 demonstrates observed enhancement of cellular aggregation during interspecies hydrogen transfer (FIG. 12) and led to the conclusion that syntrophic interactions of C. saccharolyticus with M. thermoautotrophicus influence growth dynamics and EPS production of C. saccharolyticus, which will result in more rapid hydrolysis of cellulose.

The identification (Example 7) and confirmation (Example 8) of the upregulation of genes involved in aggregation and catabolism during lignocellulose degradation may also be performed. One embodiment of the invention relates to a method of identifying a target gene for optimizing production of biomethane from cellulosic or lignocellulosic biomass, the method comprising comparing gene expression by Caldicellulosiruptor saccharolyticus in a consortium with an acetate-oxidizing thermophile and a thermophilic methanogen, wherein a change in expression of a gene is indicative of its role in production of biomethane from cellulosic biomass.

As shown in Examples 9 and 10 below, the consortium was tested in both single chamber and multi-chamber systems, to determine the optimized operating parameters for maximum productivity of the system.

Example 9 provides examination of a single chamber, continuous flow reactor. The temperature was maintained at 65-70° C. and pH 7.0. As the reactor functions, samples of the culture broth were tested to examine feedstock consumption.

Example 10 provides examination of a multi-chamber, continuous flow reactor.

When cellulose is converted to methane, an equimolar amount of CO₂ is formed (3 moles each of CH₄ and CO₂ per glucose equivalent). The consumption of cellulose is a carbon neutral process, but the CO₂ is a contaminant that reduces the value of the fuel. Therefore to increase the purity of the fuel to that of natural gas the CO₂ must be removed. A possible solution is to add an electromethanogenic component to the system.

Electromethanogenesis uses a low voltage electric current as an electron donor and a methanogen as a biocatalyst at a cathode to reduce CO₂ to methane. Cheng et al. (Cheng, S., D. Environ. Sci. Technol. (2009) 43:3953-3958) demonstrated electromethanogenesis with 1V applied to a bioelectrochemical cell and a mixed microbial community at the cathode. It is not entirely clear whether hydrogen was an intermediate in this process since a small amount of hydrogen was detected by Cheng and the voltage applied is enough to reduce protons at the cathode (but not enough to generate oxygen at the anode) even without a good catalyst (e.g. Pt). Regardless of the mechanism, the methanogen will keep the hydrogen partial pressure low, which will help pull the reaction toward CO₂ consumption and methane production. Therefore, if biogas from a cellulose-to-methane reactor was passed through an electromethanogenic cell, then the CO₂ would be converted into more biomethane fuel (FIG. 2). Methanosarcina or Methanococcus are listed at the cathode in FIG. 2. These are hydrogen consuming, mesophilic methanogens and similar organisms might work. However, direct electron transfer from the cathode by the biocatalyst may facilitate the process and these are good candidates due to their ability to donate electrons to external electron acceptors (Bond, D. R., et al., Environ Microbiol (2002) 4:115-24). Alternatively, if the off-gas from the thermophilic bioreactor is too hot, a thermophile such as M. thermolithotrophicus or M. thermoautotrophicus could be used.

A mixed electromethanogenic microbial community may also be considered. In this case an electrode oxidizing bacterium, e.g. a Geobacter sp. or Thermincola sp. may grow syntrohpically with a methanogenic archaeon. The electrode oxidizer would accept electrons and transfer them on to the methanogen as hydrogen and the methanogen would then reduce CO₂ to methane.

Cheng et al. observed electromethanogenesis with 0.7 to 1.2 V applied to an electrochemical cell, but this is not enough voltage to oxidize water to oxygen, with or without Pt, even though it is enough to reduce protons to hydrogen. Therefore, the source of protons for Cheng had to be something other than water and protons associated with phosphate ions might have been the source. Supplying phosphate ions continuously would not be practical so one would have to oxidize water. To do so usually requires an expensive catalyst, but Kanan and Nocera (Kanan, M. W., et al. Sci. (2008) 321:1072-1075) recently demonstrated the use of Co/Pi solutions at an anode as a very good catalyst for water oxidation. The reaction then occurs near the theoretical minimum (1.23 V) and not closer to 2.0 V, which usually occurs due to overvoltage. Stainless steel may also serve as the electrode. These alternatives would significantly reduce the operational cost of the electromethanogenic unit. The methanogens described here will readily grow and produce methane in solutions of Co/Pi at the concentrations tested by Nocera et al. This indicates that inexpensive chemical and microbial catalysts could be used to generate protons and then reduce CO₂ to methane in an electromethanogenic cell. The oxygen that would form at the anode would be toxic to the methanogens, but this can be vented away from the methanogens at the cathode and possibly harvested as a high purity value-added product.

In one embodiment, the invention provides a consortium that includes a cellulose and hemicellulose hydrolyzing cellulolytic thermophile, an acetate-oxidizing thermophile, and a hydrogen-utilizing thermophilic methanogen in combination with an electrochemical cell, wherein the electrochemical cell is effective to further reduce CO₂ emissions from the consortium.

In one embodiment the invention provides a stable, thermophilic microbial consortium developed and examined in batch cultures and in a continuous flow bioreactor.

Cultures are also examined at the gene level, as described in Examples 7 and 8, in order to determine the molecular response of C. saccharolyticus to activity of the tri-culture.

Identification of genes through the methods of Examples 7 and 8 will identify genetic targets that may be used in future selection and engineering of biofuel producing microorganisms. Example 7 demonstrates a method for identification of regulated genes by comparative gene expression analysis of C. saccharolyticus grown alone on plant biomass and in co-culture with the hydrogenotrophic methanogenic and acetate-oxidizing syntroph. Example 8 demonstrates a method for subsequent confirmation of the role of genes by Q-RT-PCR analysis of C. saccharolyticus grown alone on plant biomass and in co-culture with the hydrogenotrophic methanogenic and acetate-oxidizing syntroph. Overall this project will transform a conventional technology, biomethane production from waste, into a new technology that will improve the conversion of next generation, dedicated energy crops into a high energy biofuel.

Oligonucleotide microarrays for transcriptome profiling will be used to identify genes expressed by C. saccharolyticus that are regulated in response to interspecies hydrogen and acetate exchange by the consortium. This molecular approach will enable identification of key indicator genes and determine their steady state levels under optimal conditions. In addition to profiling the cellular response to cell-cell interactions in the consortium, these examples provide a molecular assay for rapidly assessing the metabolic state of C. saccharolyticus growing in continuous culture. By use of such microarrays, up-regulation of hydrogenase, CO dehydrogenase, hydrolases and related catabolic genes such as oligosaccharide transporters are seen during optimal growth with the consortium. The results from this example will provide insights on the cell-to-cell interactions of the tri-culture consortium and identify targets for engineering new microbial systems for production of biofuel from cellulosics in the future.

The efficient conversion of plant biomass to biofuels in monocultures is often limited by the inability of the fermentor to simultaneously use both hexoses and pentoses, and by the production of intermediates that divert some of the potential energy away form useful biofuels and can accumulate to inhibit complete conversion. Combining C. saccharolyticus, a thermophilic homoacetogen that concurrently hydrolyzes hexoses and pentoses, with an acetate-oxidizing syntroph and hydrogen utilizing methanogen has the potential to convert plant biomass to the theoretical limit of bioenergy.

The results provided herein identify the steady state levels of intermediates between the consortium during optimal growth. The data also identify specific genes and reveal the steady state levels of specific gene products that are directly affected by the interaction of the consortium members. In one embodiment the invention provides a molecular profile for the effect of gene expression in a fermentor by a methanogenic consortium during biomethane production. Since the transcriptome has a relatively short half life, knowledge of the molecular responses to various perturbations will provide a means of monitoring the health of the bioconversion process providing a means of responding to the adverse changes in the consortium before the process is adversely affected.

On a broader scale, the methods of the invention will lead to a better understanding of the effects of cell-cell interactions in anaerobic consortia. As opposed to fermentation with monocultures, using consortia more accurately mimics biodegradative processes that occur in the environment. Understanding how the genes of fermentative bacteria such as C. saccharolyticus are affected by a methanogenic consortium will provide greater understanding of how cells interact at the molecular level to ultimately convert plant biomass to its basic constituents. The successful completion of this project will likely provide insight into strategies used by consortia to efficiently degrade biomass and has the potential to transform current concepts on conversion of plant biomass to bioenergy.

The methods of the invention contribute directly to the scientific community in the area of microbiology, particularly in the fields of microbial physiology, and cell-cell interactions, gene regulation, genomics and bioengineering. On a larger societal scale, the current proposal addresses a national mandate to discover new domestic sources of energy, reduce the national dependence on imported fossil fuels, and mitigate the threat of global warming by providing a new, more efficient process whereby methanogenesis is used to more effectively hydrolyze cellulosic biomass from renewable, non-food energy crops and produce biofuel. With recent initiatives to increase reliance on natural gas such as the New Alternative Transportation to Give Americans Solutions Act of 2009, and smaller initiatives in California and other locations, the requirement for a sustainable, carbon neutral source of methane is now being recognized. The invention provides methods as a foothold into strategies for efficient production of biomethane from plant biomass and will complement ongoing development into alternative bioenergy sources such as biodiesel and bioethanol.

The advantages and features of the invention are further illustrated with reference to the following examples, which are not to be construed as in any way limiting the scope of the invention but rather as illustrative of embodiments of the invention in a specific application thereof.

### EXAMPLE 1

### Co-Cultures

Co-Culture of C. saccharolyticus and M. thermoautotrophicus

This example provides an examination of the physiological interaction between the cellulose hydrolyzing C. saccharolyticus (DSM 8903) and a hydrogenotrophic methanogen, Methanobacter thermoautotrophicus strain AH (DSM 1053). This will determine the effect of interspecies hydrogen and acetate transfer on the cellulose hydrolyzing extreme thermophile. C. saccharolyticus will be maintained in anaerobic DSMZ 640 medium with 50 mM glucose or with 10 g/L crystalline cellulose (Avicel) at 70° C. M. thermoautotrophicus will be maintained in anaerobic DSMZ 119 medium under 2 atm 80:20 (vol:vol) headspace of H₂:CO₂ at 65° C. All cultures will be maintained in 10 ml of media in anaerobe tubes or in 50 ml of media in 150 ml serum bottles, each sealed with black butyl stoppers. Cellulose will be measured directly by dry weight after washing of the spent media and indirectly by spectrophotometric measurement of glucose equivalents after hydrolysis of residual cellulose and adding 3:5-dinitrosalicylic acid to the reducing sugars (Miller, G. Anal. Chem. (1959) 31:426-428.). Fatty acids will monitored by HPLC (Yang, S.-J., et al., Appl. Environ. Microbiol. (2009) 75:4762-4769). Dissolved hydrogen will be measured directly by stripping the dissolved gases in the medium with concentrated KOH and measuring the concentrated hydrogen by GC-FID (Robinson, J. A., et al. Appl. Environ. Microbiol. (1981)41:545-548.). The pH will be determined in aliquots with a microprobe.

To accurately determine the mass balance, the entire contents of a culture vessel must be analyzed in order to collect all residual cellulose. In this case, experiments will be prepared with replicate cultures designated for sacrifice at each time point. Otherwise, experimental cultures will be maintained throughout an experiment and multiple headspace samples for hydrogen and methane, and media samples for fatty acids will be taken. A 0.5 ml aliquot of culture broth will be extracted for nucleic acid analysis as described in Example 8 herein. Aliquots will be immediately frozen and shipped on dry ice to UMBI, or DNA/RNA will be extracted and prepared as described in Example 8 herein and the nucleic acid then shipped to UMBI. All experiments will be prepared with a minimum of three replicate cultures for each time point and maintained at 65 to 70° C. in batch for one week or until the majority of cellulose has been consumed. Each experiment will be designed so that consumption rates of cellulose are determined as are rates and yields of product formation. These values will be normalized to moles of glucose equivalents added to the media. Overall biomass production will be determined as protein, protein and cell numbers will be determined for each species using quantitative real-time PCR with specific primers for each species as described below in Example 8.

Each of C. saccharolyticus, M. thermoautotrophicus, and T. ferriacetia has been tested for growth in DSMZ 640 and 119. C. saccharolyticus and M. thermoautotrophicus were observed to grow equally well in each medium, with the exception that the concentration of sulfide ordinarily added to DSMZ 119 had to be decreased to 0.5 g/L or less to enable the growth of C. saccharolyticus. At the concentration called for in DSMZ 119 (1.5 g/L) some inhibition of C. saccharolyticus was observed. However, M. thermoautotrophicus grew well with a reduced sulfide concentration (0.5 g/L) that did not inhibit C. saccharolyticus. T. ferriacetia will also grow well in the reduced sulfide concentration (0.5 g/L).

An active M. thermoautotrophicus culture (10⁷ cells/ml) was inoculated into a culture of C. saccharolyticus (10⁹ cells/ml) actively growing on 2.5 g/L cellulose at 65° C. to allow for sufficient biotic formation of H₂:CO₂ required for the growth of M. thermoautotrophicus. The production of methane was observed within 1 day of inoculation and C. saccharolyticus continued to produce hydrogen throughout the course of the experiment.

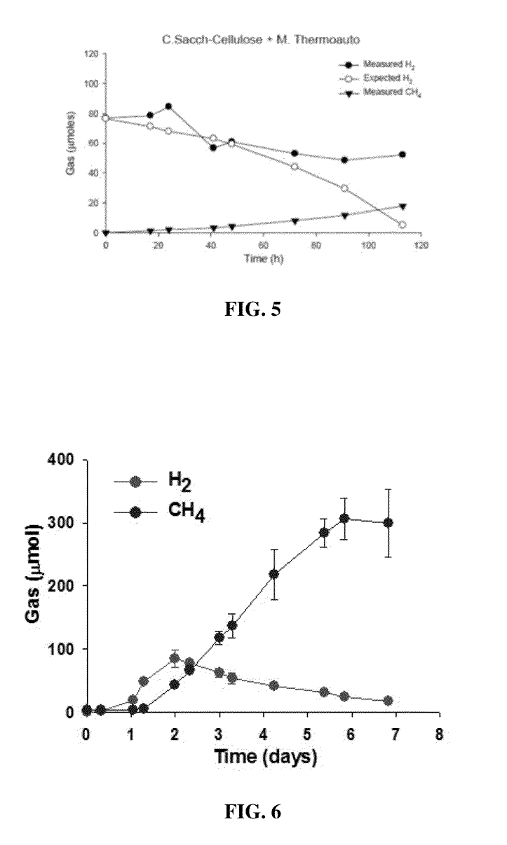

Inoculating an active M. thermoautotrophicus (10% vol/vol from a culture with 10⁸ cells/ml, sulfide concentration after inoculation ˜0.05 g/L) into a culture of C. saccharolyticus actively growing on cellulose at 65° C. resulted in the continued production of hydrogen by C. saccharolyticus and the production of methane, therefore the coculture will continue to hydrolyze cellulose and produce hydrogen while producing methane (FIG. 5).

The simultaneous inoculation of C. saccharolyticus and M. thermoautotrophicus (10% v/v each) also resulted in an initial increase of hydrogen from cellulose hydrolysis followed by the production of methane (FIG. 6). Thereafter, the established cocultures were maintained as a single inoculum throughout sequential transfers without loss of activity.

Elimination of sulfide entirely from the co-culture medium has also been tested. Maintaining a low concentration of sulfide is important for the application of this system since sulfide can be corrosive to internal combustion engines and furnaces that use biomethane.

The conversion of cellulose into methane by a co-culture of Caldicellulosiruptor saccharolyticus and Methanothermobacter thermoautotrophicus was evaluated by the following procedure: Cellulose powder (2.5 g/L) was added to 50 ml culture bottles inoculated with the co-culture. At the end of the experiment the cellulose was no longer visible, was not measurable by weight, and no reducing sugars were detected. This demonstrates that cellulose was consumed and methane was produced by the co-culture. The culture maintained a very low hydrogen concentration, and converted the hydrogen into methane.

TABLE 5
Products of cellulose fermentation (mmoles/g of cellulose) after 7 days.

| | CH₄ | H₂ | Acetate | Lactate | Formate |
|---|---|---|---|---|---|
| Co-culture | 1.6 ± 0.3 | 0.13 ± 0.01 | 8.0 ± 0.3 | 3.2 ± 0.7 | 0.9 ± 0.2 |

Co-culture: C. saccharolyticus + M. thermoautotrophicus

FIG. 7 shows the production of hydrogen, or hydrogen equivalents produced as methane, from C. saccharolyticus alone, the co-culture, and the tri-culture (Example 3) when incubated with 2.5 g of cellulose powder. The data indicate that the triculture more rapidly, and to a higher yield, converted cellulose into hydrogen-equivalents (methane) than the co-culture, which more rapidly converted cellulose into hydrogen equivalents than the solitary C. saccharolyticus. Overall the data indicate that celluose fermentation and production of useful products (fuels such as hydrogen or methane) is more rapidly and efficiently accomplished with the co-culture and tri-culture.

Co-Culture of C. saccharolyticus and an Acetate-Oxidizing Bacterium

Examining a co-culture of C. saccharolyticus with just an acetate-oxidizer is more problematic since there would not be an electron (H₂) acceptor for the acetate oxidizer. However, an artificial mechanism can be used to observe such a co-culture, i.e. incubation of the microorganisms in a bioelectrochemical cell. To do this will require that the acetate-oxidizing microorganism be capable of using an electrode as an electron acceptor. The inventors have demonstrated that that T. ferriacetica will do this while oxidizing acetate (Marshall, C. W., and H. D. May, Energy Environ. Sci. (2008) 2:699-705.). Using similar methodology the inventors have generated an electric current with a co-culture of C. saccharolyticus and T. ferriacetica in a bioelectrochemical cell with cellulose as the sole carbon and energy source. This allows for real-time monitoring of the metabolism of the co-culture through analysis of an electric current, and it maintains a culture dependent on interspecies acetate transfer. Otherwise, analysis of the culture and fermentation products remains the same.

Co-Culture of M. thermoautotrophicus and T. ferriacetica

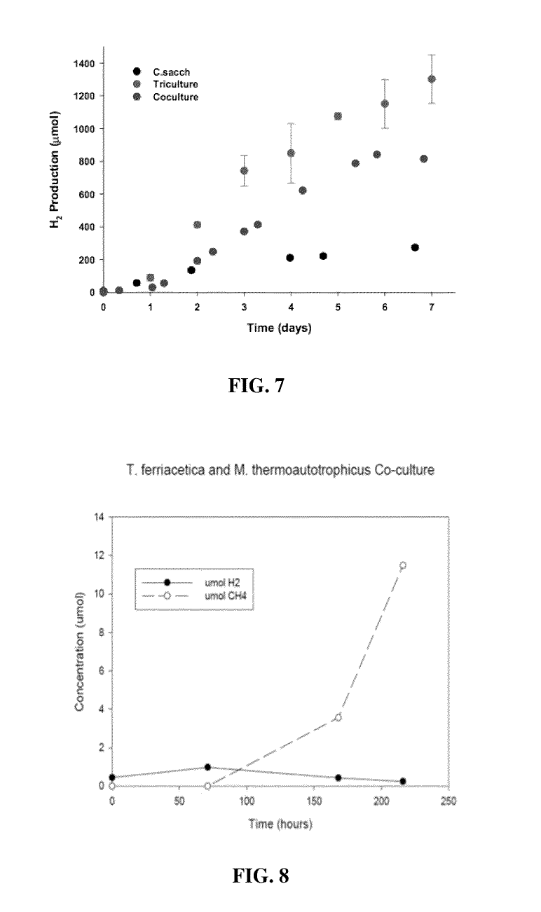

The methodology and conditions described above were also used for a co-culture of M. thermoautotrophicus and T. ferriacetica to test for the ability of the acetogen to reverse its catabolism and grow as an acetate-oxidizer via inter-species hydrogen exchange. FIG. 8 shows that when grown in medium containing only acetate the co-culture produced methane. Since M. thermoautotrophicus does not catabolize acetate for methanogenesis, the results show for the first time that T. ferriacetica grows as an acetate-oxidizer in the presence of a hydrogen utilizing methanogen.

### EXAMPLE 2

### Enhancement of Cellulose Hydrolysis by C. saccharolyticus Grown-in Co-Culture with M. thermoautotrophicus

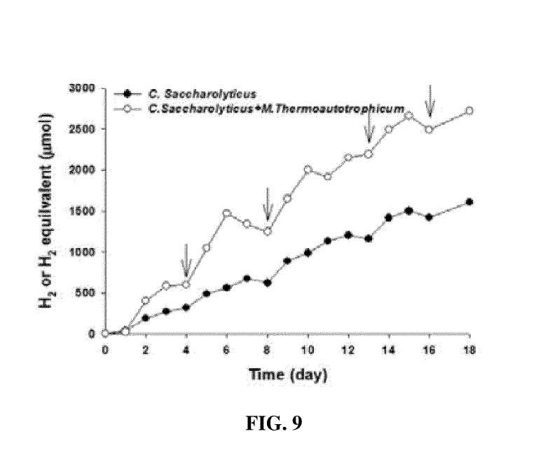

Co-culturing C. saccharolyticus with M. thermoautotrophicus significantly improved the rate and yield of cellulose hydrolysis in comparison with a monoculture of C. saccharolyticus. When grown on cellulose in semibatch reactor, the production rate and yield of hydrogen, which was completely converted to methane in the coculture, were 2× greater than that from a monoculture of C. saccharolyticus (FIG. 9). The yield of hydrogen (22.7 mmol H₂/g cellulose) in the coculture reached 89% of the theoretical maximum (24.7 mmol H₂/g cellulose calculated using glucose units and Thauer's limit), while that of the monoculture was 48% of the theoretical maximum (12.0 mmol H₂/g cellulose).

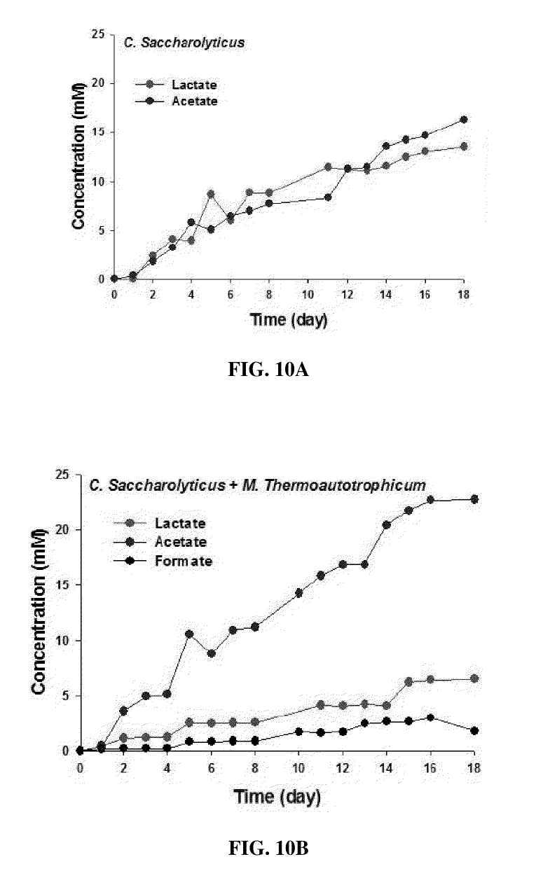

Moreover, co-culturing C. saccharolyticus with M. thermoautotrophicus affected the distribution of fermentation products besides H₂ by producing significantly less lactate and more acetate than the monoculture, which results in a greater energy yield (ATP) for C. saccharolyticus (FIG. 10). M. thermoautotrophicus was responsible for diverting electron flow away from pyruvate reduction to lactate resulting in more oxidized products (acetate and formate) and more H₂ by maintaining low hydrogen partial pressure.

### EXAMPLE 3

### Tri-Culture of C. saccharolyticus, M. thermoautotrophicus and T. lettingae

The methodology and conditions described in Example 1 are applied here but with the addition of the acetogenic syntroph T. lettingae (DSM 14835). T. lettingae will be maintained in anaerobic DSMZ 664 medium at 65° C. with glucose or acetate as carbon sources. In the latter case, thiosulfate will be added as an electron acceptor. In addition, the yeast extract of the medium will be reduced to 0.5 g/L (proven sufficient by Balk et al. (Balk, M., et al. Int. J. Syst. Evol. Microbiol. (2002)52:1361-1368.). T. lettingae has been shown to grow in co-culture with M. thermoautotrophicus strain ΔH with acetate as the sole carbon source (Balk, M., et al.). Each microorganism is capable of growing in all DSMZ media described and has a similar temperature, pH and salinity optimum. Cellulose consumption, product formation, methane yields and rates, and specific growth rates will be assayed as described in Example 1. Specific growth rate of T. lettingae will be made indirectly through quantitative PCR with primers specific to the 16S rRNA of this microorganism.

DSMZ 664 medium contains cysteine and sulfide (1.5 g/L) to be added as reductants, just as in DSMZ 119 for M. thermoautotrophicus. As noted above, sulfide at this concentration inhibits the growth of C. saccharolyticus. The metabolic activities of dense co-culture will also provide adequate reducing potential without addition of high amounts of sulfide.

The tri-culture has also been performed under the same conditions described above, with Thermincola ferriacetica as the acetate-oxidizing thermophile.

The conversion of cellulose into methane by a tri-culture of C. saccharolyticus, M. thermoautotrophicus, and Thermincola ferriacetica was evaluated by the following procedure: Cellulose powder (2.5 g/L) was added to 50 ml culture bottles inoculated with the co-culture (Example 1) or tri-culture (data are from triplicate cultures). At the end of the experiment the cellulose was no longer visible, was not measurable by weight, and no reducing sugars were detected. This demonstrates that cellulose was consumed and methane was produced by the co-culture and the tri-culture. Both cultures maintained a very low hydrogen concentration, lowest by the tri-culture, and converted the hydrogen into methane. The data also indicate that the ratio of methane to fatty acids was increased with the tri-culture.

TABLE 6
Products of cellulose fermentation (mmoles/g of cellulose) after 7 days.

| | CH₄ | H₂ | Acetate | Lactate | Formate |
|---|---|---|---|---|---|
| Tri-culture | 2.6 ± 0.3 | 0.03 ± 0.01 | 4.7 ± 0.4 | 2.0 ± 0.4 | <0.1 |

Tri-culture: C. saccharolyticus + M. thermoautotrophicus + T. ferriacetica

FIG. 7 shows the production of hydrogen, or hydrogen equivalents produced as methane, from C. saccharolyticus alone, the co-culture (Example 1), and the tri-culture when incubated with 2.5 g of cellulose powder. The data indicate that the triculture more rapidly, and to a higher yield, converted cellulose into hydrogen-equivalents (methane) than the co-culture, which more rapidly converted cellulose into hydrogen equivalents than the solitary C. saccharolyticus. Overall the data indicate that celluose fermentation and production of useful products (fuels such as hydrogen or methane) is more rapidly and efficiently accomplished with the co-culture and tri-culture.

### EXAMPLE 4

### Comparison of Methane Yields from Treated and Untreated Switchgrass

The experiments in Examples 1, 2, and 3 were restricted to the fermentation of pure cellulose in order to establish the effect of the tri-culture on cellulose hydrolysis. However, it is important to evaluate both treated and untreated cellulose.

In one embodiment, switchgrass may be used as the cellulosic biomass. The switchgrass is grown in South Carolina and baled as straw. The straw will be milled to particles of ˜2-4 mm in length and 1-2 mm in diameter and then added to media as described for pure cellulose in Examples 1, 2, and 3. The methodology and approach thereafter is identical except there will be a hemicellulose fraction and a residual, non-degraded lignin fraction. Therefore culture broth for the production and consumption of specific sugars (glucose, xylose, arabinose, etc.) will be examined, in addition to fatty acids and hydrogen. This will be achieved by HPLC equipped with a Shimadzu refractive index detector. Otherwise, the examination of hydrolysis and fermentation products remains the same.

Additionally the productivity of the tri-culture with switchgrass pre-treated in a batch process called soaking aqueous ammonia (SAA) (Isci, A., et al., Appl. Biochem. Biotechnol. (2008) 144:69-77.) was evaluated. In this case, switchgrass will be soaked in 30% ammonia hydroxide for 10 days at 25° C. or 12 h at 60° C. The ammonia and lignin (75%) are washed away with water. The resulting mixture contains more exposed cellulose and hemicellulose with minimal if any inhibitory compounds.

C. saccharolyticus has been grown on this pretreated material.

Experiments are in progress with both pretreated switchgrass and non-treated switchgrass, using a co-culture of C. saccharolyticus and M. thermoautotrophicus or a tri-culture of C. saccharolyticus, M. thermoautotrophicus, and T. ferriacetica. Production of methane has been observed.

### EXAMPLE 5

### Enhancement of Switchgrass Hydrolysis by C. saccharolyticus Grown with M. thermoautotrophicus

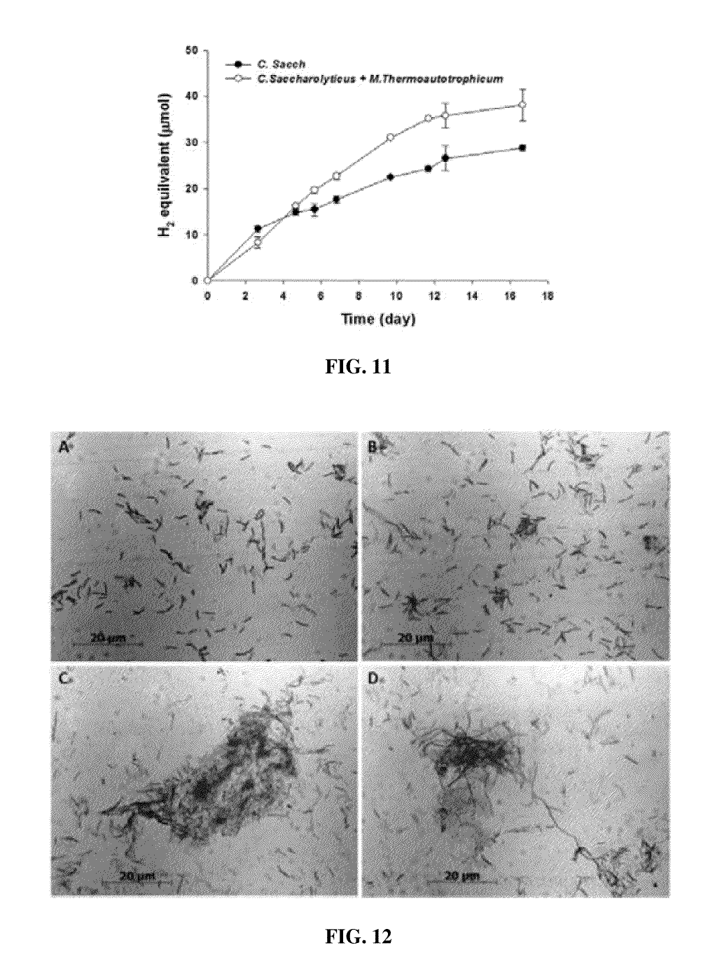

When untreated switchgrass was used as a substrate, the yield of hydrogen was greater in the coculture of C. saccharolyticus and M. thermoautotrophicus in comparison with what was produced by a monoculture of C. saccharolyticus (FIG. 11). Overall comparison of the rate and yield of hydrogen production between the monoculture and the coculture when they are grown with switchgrass (FIG. 11) is comparable to that when grown with pure cellulose as model compound (FIG. 10), suggesting that interspecies hydrogen transfer promotes greater hydrolysis of lignocellulosics. Moreover, this process can be improved by using switch grasses pretreated with chemicals (SAA or sulfuric acid), physical machine (ball milling) or enzymes prior to microbial hydrolysis.

### EXAMPLE 6

### Enhancement of Cellular Aggregation During Interspecies Hydrogen Transfer

Syntrophic cell-cell interactions can have significant impact on the microbial physiology of microorganisms compared with cells grown in monoculture. When the thermophiles T. maritima and M. jannaschii were grown in coculture, the cell density of T. maritima increased with concomitant formation of EPSmediated cell aggregates. The aggregation appears to facilitate interspecies hydrogen transfer, possibly by maintaining critical cell-cell proximity. Similarly, the formation of stable cellular aggregates were observed in the Gram-stained C. saccharolyticus when it was grown in coculture with M. thermoautotrophicus (FIG. 12). In the monoculture, some cells clustered together near cellulose crystals but the size and the number of aggregates are much smaller than those in the coculture. In view of these observations, it was hypothesized that syntrophic interactions of C. saccharolyticus with M. thermoautotrophicus influence growth dynamics and EPS production of C. saccharolyticus, which will result in more rapid hydrolysis of cellulose. Therefore, genes involved in aggregation as well as catabolism should be upregulated during lignocellulose degradation.

### EXAMPLE 7

### Identification of Regulated Genes

Microarrays generated by maskless photolithography will be obtained from NimbleGen Systems. Each array will include 95% of 2679 orfs in the C. saccharolyticus DSM 8903 genome (Genbank Accession No. CP000679) each represented by an average of 17 unique probes in triplicate. C. saccharolyticus will be harvested in mid-exponential growth from batch cultures grown both with and without M. thermoautotrophicus and/or T. lettingae. Total RNA will be isolated as described elsewhere (Zhang, W., et al., J Ind Microbiol Biotechnol (2006) 33:784-90.). Labeling with biotinylated ddATP, hybridization, washing and scanning will be performed as described previously (Nuwaysir, E. F., et al., Genome Res. (2002) 12:1749-1755.). Spot signal intensities will be adjusted by subtracting local background, and a two-sided t test will be performed to assess whether the signal is significantly different from that of the background. The significance of the log ratio calculated from normalized process signals will be assessed by computing the most conservative log ratio error and significance value (P value), using a standard error propagation algorithm and a universal error model (Rosetta Biosoftware). After removal of outliers, the final log ratio, fold change of gene expression, log ratio error, and P value for a gene will be calculated as arithmetic means of all probe values for that gene. Based on differential expression of genes in C. saccharolyticus grown in pure culture and in a consortium, genes that are upregulated in response to growth on plant biomass in the consortium will be identified.

### EXAMPLE 8

### Confirmation of Gene Regulation

To confirm the role of indicator genes specifically up-regulated during optimal growth, RNA will be extracted from C. saccharolyticus exponential phase cells grown on biomass as a pure culture and in a consortium with the RNeasy kit (Qiagen) following the manufacturer's instructions. RNA (50 ng) will be used as template for each qRT-PCR using the iScript one-step RT-PCR kit (Bio-Rad) following the manufacturer's instructions. The primers used in the qRT-PCR reactions will be designed for target genes identified in DNA microarrays using Beacon Designer software (PREMIER Biosoft International). To compare the fold differences in expression, the C(t) values for each reaction will be normalized to the C(t) value for a housekeeping gene such as ffh, glnA, gyrA, proC, recA, rho, rplI, rplQ, topA, tsx, malE (Takle, G., I., et al. BMC Plant Biol. 7:1-9 (2007).). Initially the stability of the gene products will be confirmed by quantifying them under different growth conditions (i.e., with and without consortia members) with the DNA microarray. Once a reference gene has been confirmed the fold differences will be calculated using the formula: fold difference=2^{(ΔΔ(C(t)))}, where ΔΔC(t) is the difference in the normalized C(t) values of pure versus consortium cultures (Schmittgen, T. D., et al., Nat Protoc (2008) 3:1101-1108.). Results of these experiments with C. saccharolyticus will confirm the effects of interspecies hydrogen and acetate exchange on specific genes during growth in the consortium. The method will also establish target genes for monitoring optimal growth. Unlike monitoring cell numbers or proteins, which can remain for various periods ranging from hours to days after an adverse permutation has taken effect, prokaryotic mRNA has a relatively short half-life in minutes, which will enable us to detect immediate changes in the consortium associated with sub-optimal conditions. The C(t) values of 16S rRNA genes will also be established with selective primers for all 3 member of the consortium. This will enable quantification of the number of cells for each consortium member during growth in mixed culture.

It is expected that genes involved in oligosaccharide hydrolysis, NADH and ferredoxin linked hydrogenases and CO dehydrogenase will be up-regulated in response to the tri-culture, and effects on other related genes such as oligosaccharide transporters may become apparent. In addition, up-regulation of other genes such as metal transporters might be observed that would enable identification of potentially limiting factors of the process. There is also the possibility that genes related to hydrolysis, acetogenesis or hydrogenesis may be expressed constitutively, but some degree of attenuation detectable with the microarrays is to be expected. Successful completion of the proposed DNA array hybridization experiments will depend on the ability to harvest RNA from C. saccharolyticus. Generally prokaryotic mRNA has a short half-life and is susceptible to degradation.

In addition to providing a monitoring assay for optimal operating parameters for the triculture, identification of genes specifically expressed during optimal biomass conversion will provide targets to screen potential biocatalysts for bioconversion in other systems. For example, a specific carbohydrate transporter or hydrolase may be discovered to be up-regulated in response to the presence of the consortium. Screening for this transporter or hydrolase, or detection of multiple copies among other biomass hydrolyzers, would provide the means to identify hydrolyzers that could work more rapidly or efficiently in the consortium. Genes linked to methanogenic bioconversion could also serve as targets for designing metabolic pathways for the production of biochemicals and next generation biofuels from a variety of feedstocks.

### EXAMPLE 9

### Optimize Operating Parameters of the Tri-Culture in a Single Chamber, Continuous Flow Reactor

The culture will be established in a jacketed 20-L Bioflo IV bioreactor (New Brunswick Scientific) at a working volume of 16 L. Temperature will be maintained at 65 to 70° C. and pH at 7.0 by the automatic addition of NaOH. The contents will be continuously stirred at a rate that suspends all solids, and all vessels including media reservoirs will be sparged continuously with 180 L h⁻¹ of N₂. The reactor will be started in batch with milled and untreated switchgrass or pretreated switchgrass at 5 g dry weight per liter in media used for Examples 1-5. Increasing loads of feedstock will be tested. Once signs of growth and activity have been observed (expected in less than a day) continuous supply of media will start at a dilution rate of D=0.05 h⁻¹. Dilution rates will be increased until productivity is observed to decrease. Steady state will be considered achieved when methane production rates remain constant after exchange of 5 working volumes. Culture broth from within the reactor will be sampled for examination of feedstock consumption and levels of fermentation intermediates including dissolved hydrogen. Hydrogen, CO₂ and CH₄ will be measured also in the off gas.

DNA or RNA will be extracted form subsamples to determine changes in target gene expression and population dynamics through 16S rRNA gene analysis. Baseline C(t) values of target genes will be established during steady state optimal growth of C. saccharolyticus in the chemostat tri-culture. One advantage of the continuous-flow anaerobic culture system is that a single growth parameter can be manipulated. Once the baseline expression level for the target genes under steady state conditions has been established, the experiment will be repeated under different non-ideal permutations and compare the changes in C(t) values with 16S rRNA (cell numbers), hexose and pentose hydrolysis, dissolved hydrogen and acetate concentrations and methane production. Permutations will include grown without the syntroph, without the methanogen, at different temperatures (70° C.+/−5, 10° C.) and pH (pH 6.0-8.0 @ 0.5 intervals) Results will quantify the interactive effect of different permutations on rates of hydrolysis and methane formation, steady state levels of intermediates, steady state ratios of triculture members. Establishing the response of target genes to these permutations will provide a tool for rapid assessment (ca. 4 hr) on the physiological state of the culture. Coupling of the continuous-flow system with real-time qRT-PCR will enable us to study the effects of environmental changes on cell-cell interactions by measuring the expression of specific bacterial genes. For example, if qRT-PCR indicated that the ferredoxin linked hydrogenase was down regulated, and qRT-PCR confirmed that M. thermoautotrophicus numbers were below optimum, it would be possible to intervene by adding more methanogen before the culture stalled. Since mRNA has a short half-life, restoration of optimal levels of hydrogenase mRNA would confirm that the culture was stabling long before detection of product or intermediates

All of the needed assays are ready as described for the batch experiments. However, the method of applying additional switchgrass will have to be tested during the development of the continuous flow reactor. The switchgrass will be milled to particles of ˜2-4 mm by 1-2 mm, treated or untreated, so it anticipated that a sufficient supply may be transferred by stirring the supply reservoir and pumping the contents through a tube with a diameter of 1 cm. The resulting concentration of feedstock will then have to be measured within the bioreactor. Switchgrass is poor in N, but the ammonium ion added to the medium will be sufficient for these studies. For the development of a true application, either the ammonium salts must be added or some of the ammonia from the SAA may be used to supplement the process. However, C. saccharolyticus and M. thermoautotrophicus possess the capability to fix N₂, which may lower the need for an additional N source.

### EXAMPLE 10

### Optimize Operating Parameters of the Tri-Culture in a Multi-Chamber, Continuous Flow Reactor

As noted herein, the ability of T. lettingae to consume sugars may delay or inhibit oxidation of acetate to CO₂ and H₂. Although this may be overcome with time in batch reactors or by substituting T. ferriacetica for T. lettingae, maximum productivity may require the use of a two-stage bioreactor. The approach and methodology will be very similar to what is described in Example 9, but now the system will be divided into two sequential reactors, the first inoculated with C. saccharolyticus and M. thermoautotrophicus and the second with T. lettingae and M. thermoautotrophicus. The effluent from the first reactor will be transferred to the second reactor. Cellulose consumption, and the generation of sugars, fatty acids, H₂, CO₂ and methane will be measured in the culture broth and off gas of the first reactor, and acetate, CO₂, H₂ and CH₄ will be measured in the culture broth and off gas of the second reactor. DNA or RNA will be extracted form subsamples to determine changes in target gene expression and population dynamics through 16S rRNA gene analysis.

Continuous flow from one reactor to the other will be attempted, but the system may need to be operated in semibatch mode to achieve maximum productivity. For the latter case, once the concentration of hexoses and pentoses are sufficiently low the contents of the first reactor will be transferred to the second reactor and the first one re-filled with fresh media and feedstock. Alternatively, a semi-permeable selective membrane may be used to filter the effluent from reactor one to reactor two. This membrane would separate compounds based on size and/or charge (e.g. a anion exchange membrane). In this way the sugars would be retained in reactor one and acetate be passed on to reactor two. Control of flow into and out of each reactor must be maintained separately for semi-batch operation and for continuous flow, and in the latter case it may be necessary to operate at two different dilution rates.

While the invention has been has been described herein in reference to specific aspects, features and illustrative embodiments of the invention, it will be appreciated that the utility of the invention is not thus limited, but rather extends to and encompasses numerous other variations, modifications and alternative embodiments, as will suggest themselves to those of ordinary skill in the field of the present invention, based on the disclosure herein. Correspondingly, the invention as hereinafter claimed is intended to be broadly construed and interpreted, as including all such variations, modifications and alternative embodiments, within its spirit and scope.

What is claimed is:

1. A thermophilic microbial consortium for conversion of cellulosic or lignocellulosic biomass to methane, the consortium comprising:
a) a cellulolytic thermophile;
b) an acetate-oxidizing thermophile effective to oxidize acetate to carbon dioxide and hydrogen; and
c) a hydrogen-utilizing thermophilic methanogen.

2. The thermophilic microbial consortium of claim 1, wherein the cellulolytic thermophile is selected from the group consisting of Caldicellulosiruptor saccharolyticus and Caldicellulosiruptor bescii.

3. The thermophilic microbial consortium of claim 1, wherein the acetate-oxidizing thermophile is selected from the group consisting of Thermatoga lettingae and Thermincola ferriacetica.

4. The thermophilic microbial consortium of claim 1, wherein the thermophilic methanogen is selected from the group consisting of Methanothermobacter thermoautotrophicus, Methanobacterium thermoaggregans, Methanothermobacter defluvii, Methanothermobacter marburgensis, and Methanothermobacter wolfei.

5. The thermophilic microbial consortium of claim 1, wherein a), b), and c) are thermophilically active at a temperature in a range of from about 60° C. to about 75° C.

6. A system for the conversion of cellulosic or lignocellulosic biomass to methane comprising:
a) a thermophilic microbial consortium comprising a cellulolytic thermophile, an acetate-oxidizing thermophile effective to oxidize acetate to carbon dioxide and hydrogen, and a hydrogen-utilizing thermophilic methanogen; and
b) an electromethanogenic electrochemical cell.

7. The system of claim 6, wherein the cellulolytic thermophile is selected from the group consisting of Caldicellulosiruptor saccharolyticus and Caldicellulosiruptor bescii.

8. The system of claim 6 wherein the acetate-oxidizing thermophile is selected from the group consisting of Thermatoga lettingae and Thermincola ferriacetica.

9. The system of claim 6, wherein the thermophilic methanogen is selected from the group consisting of Methanothennobacter thermoautotrophicus, Methanobacterium thermoaggregans, Methanothermobacter defluvii, Methanothermobacter marburgensis, and Methanothermobacter wolfei.

10. The system of claim 6, wherein the cellulolytic thermophile, the acetate-oxidizing thermophile, and the thermophilic methanogen are thermophilically active at a temperature in a range of from about 60° C. to about 75° C.

11. The system of claim 6, wherein the consortium is contained within a single chamber continuous flow reactor.

12. The system of claim 6, wherein the consortium is contained within a multi-chamber continuous flow reactor.

13. The system of claim 12, wherein a first chamber contains a cellulolytic thermophile and a thermophilic methanogen and a second chamber contains an acetate-oxidizing thermophile and a thermophilic methanogen.

14. A method of converting lignocellulosic biomass to methane, comprising exposing the lignocellulosic biomass to a thermophilic microbial consortium comprising a cellulolytic thermophile, an acetate-oxidizing thermophile effective to oxidize acetate to carbon dioxide and hydrogen, and a hydrogen-utilizing thermophilic methanogen, under conditions effective for microbial action on the lignocellulosic biomass to produce lignin, CO₂ and CH₄.

15. The method of claim 14, further comprising converting the CO₂ to methane.

16. The method of claim 15, wherein said converting is carried out with use of an electromethanogenic electrochemical cell.

17. The method of claim 14, wherein the cellulolytic thermophile is selected from the group consisting of Caldicellulosiruptor saccharolyticus and Caldicellulosiruptor bescii.

18. The method of claim 14, wherein the acetate-oxidizing thermophile is selected from the group consisting of Thermatoga lettingae and Thermincola ferriacetica.

19. The method of claim 14, wherein the thermophilic methanogen is selected from the group consisting of Methanothennobacter thermoautotrophicus, Methanobacterium thermoaggregans, Methanothermobacter defluvii, Methanothermobacter marburgensis, and Methanothermobacter wolfei.

20. The method of claim 14, wherein the cellulolytic thermophile, the acetate-oxidizing thermophile, and the thermophilic methanogen are thermophilically active at a temperature in a range of from about 60° C. to about 75° C.

21. The method of claim 14, wherein the lignocellulosic biomass comprises switchgrass or poplar.

22. The method of claim 14, further comprising pretreatment of the lignocellulosic biomass prior to addition to the reactor.

23. The method of claim 22, wherein pretreatment comprises soaking in aqueous ammonia (SAA).

24. The method of claim 14, wherein the reactor comprises a single chamber continuous flow reactor.

25. The method of claim 14, wherein the reactor comprises a multi-chamber continuous flow reactor.

26. The method of claim 25, wherein a first chamber contains a cellulolytic thermophile and a thermophilic methanogen and a second chamber contains an acetate-oxidizing thermophile and a thermophilic methanogen.

27. A method comprising microbial conversion of cellulosic or lignocellulosic biomass to methane, wherein the microbial conversion comprises microbial action by a cellulolytic thermophile, an acetate-oxidizing thermophile and a hydrogen-utilizing methanogenic thermophile.
